(19) United States
(12) Patent Application Publication
(10) Pub. No.: US 2011/0024518 A1
(43) Pub. Date: Feb. 3, 2011

(54) REFILL FRAGRANCE CARTRIDGE FOR AIR FRESHENER UNITS

(75) Inventor: Christian Weaver, Eagle Mountain, UT (US)

Correspondence Address: ALAN J. HOWARTH, P.O. BOX 1909, SANDY, UT, 84091-1909, US

(73) Assignee: F-MATIC, INC., Lehi, UT (US)

(21) Appl. No.: 12/693,295

(22) Filed: Jan. 25, 2010

Related U.S. Application Data
(60) Provisional application No. 61/147,004, filed on Jan. 23, 2009.

(51) Int. Cl. A61L 9/04

(52) U.S. Cl. 239/34

(57) ABSTRACT

A container for containing an air freshener, comprising: (a) a first cup adapted to contain the air freshener, having a closed bottom and a first open top exposing the air freshener to the air; (b) a removable first seal covering the first open top to prevent loss of the air freshener prior to use; and (c) an attachment member on the closed bottom for connecting with a second cup to remove a second seal covering a second open top of the second cup.

A method of using a first container to remove a cover on a second container, comprising: (a) contacting a bottom of the first container with a top of the second container; (b) rotating the first container relative to the second container, so that attachment means on the first container attaches to the cover of the second container; (c) removing the first container from the second container, thereby removing the cover from the second container.

### CROSS-REFERENCE TO RELATED APPLICATIONS

This application claims the benefit of U.S. Provisional Application No. 61/147,004, filed Jan. 23, 2009, which is hereby incorporated by reference herein in its entirety, including but not limited to those portions that specifically appear hereinafter, the incorporation by reference being made with the following exception: In the event that any portion of the above-referenced provisional application is inconsistent with this application, this application supercedes said above-referenced provisional application.

### STATEMENT REGARDING FEDERALLY SPONSORED RESEARCH OR DEVELOPMENT

Not applicable.

### BACKGROUND OF THE INVENTION

Air freshener dispensers are often located in public places such as in public restrooms. These dispensers may use containers of pressurized liquid freshener, actuated by depressing a spray head or opening a valve. Another type of air freshener uses a fan driven dispenser, wherein the air freshening material is disposed in an open container of liquid or gel. A fan causes air to blow across or onto the material, thereby vaporizing part of the freshening material and dispensing the vapor into a room. The material is contained in a cup or cartridge or other similar type of container that may be placed in the housing of the dispenser adjacent to the fan.

Typically gel-based air freshener compositions have a high perfume content that makes them subject to rapid evaporation rates. Accordingly, in air freshener dispensers, cartridges containing such gels must be periodically replaced. Because of the gel volatility, it is important that the tops of cartridges containing the gels be tightly sealed at the time of manufacture and remained sealed until used. At the time of use, it is advantageous that the cartridges be easily and safely opened for positioning in a dispenser.

The cartridge covers are tightly sealed, typically with aluminum foil or a metal lid. Accordingly, removal of the sealed cover presents a major safety problem. Manual removal of a seal may be difficult and time consuming. Further, manual removal of a aluminum foil or a metal lid requires exerting significant force, that may cause slipping and/or injury to the user. The use of knives or other cutting devices to remove aluminum foil or a metal lid also raises the risk of injury to the user.

### BRIEF SUMMARY OF THE INVENTION

A container for containing an air freshener, comprising: (a) a housing having a wall and a base for containing the air freshener, having an opening for exposing the air freshener to the air; (b) a removable cover disposed over the opening; and (c) contacting means on the housing adapted to contact an aperture of another container having a cover device over an aperture and adapted to remove the cover device from the aperture.

A container for containing an air freshener, comprising: (a) a first cup adapted to contain the air freshener, having a closed bottom and a first open top exposing the air freshener to the air; (b) a removable first seal covering the first open top to prevent loss of the air freshener prior to use; and (c) an attachment member on the closed bottom for connecting with a second cup to remove a second seal covering a second open top of the second cup.

A method of using a first container to remove a cover on a second container, comprising: (a) contacting a bottom of the first container with a top of the second container; (b) rotating the first container relative to the second container, so that attachment means on the first container attaches to the cover of the second container; (c) removing the first container from the second container, thereby removing the cover from the second container.

A container for containing an air freshener, comprising: (a) a cup adapted to contain the air freshener, having a closed bottom and an open top exposing the air freshener to the air; (b) a seal over the open top to seal the air freshener from evaporation or vaporization until ready for use; and (c) a plurality of members disposed in the cup to provide for additional surface area for the air freshener to evaporate or vaporize after the seal has been removed.

### BRIEF DESCRIPTION OF THE SEVERAL VIEWS OF THE DRAWINGS

In the detailed description that follows, reference will be made to the following Figures, in which:

FIG. 1 is a prior art exploded view of an air freshener unit;

FIG. 2 is a perspective view of two refill cartridges according to one embodiment of the present invention;

FIG. 3 is a top perspective view of a depleted cup according to one embodiment of the present invention;

FIG. 4 is a bottom perspective view of the cup shown in FIG. 3;

FIG. 5 is a partial close-up view of a portion of the bottom perspective view of the cup shown in FIG. 4;

FIG. 6 is a perspective view of the depleted cup shown in FIG. 2, disposed above a refill cup of like dimensions;

FIG. 7A is a perspective view of the depleted cup and refill cup of FIG. 6 in mated position;

FIG. 7B is a partial close-up view of a portion of the perspective view shown in FIG. 7A; and

FIG. 8 is a perspective view of the depleted cup and refill cup of FIG. 6 in separated position after removal of a foil seal.

### DETAILED DESCRIPTION

Before the present apparatus and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

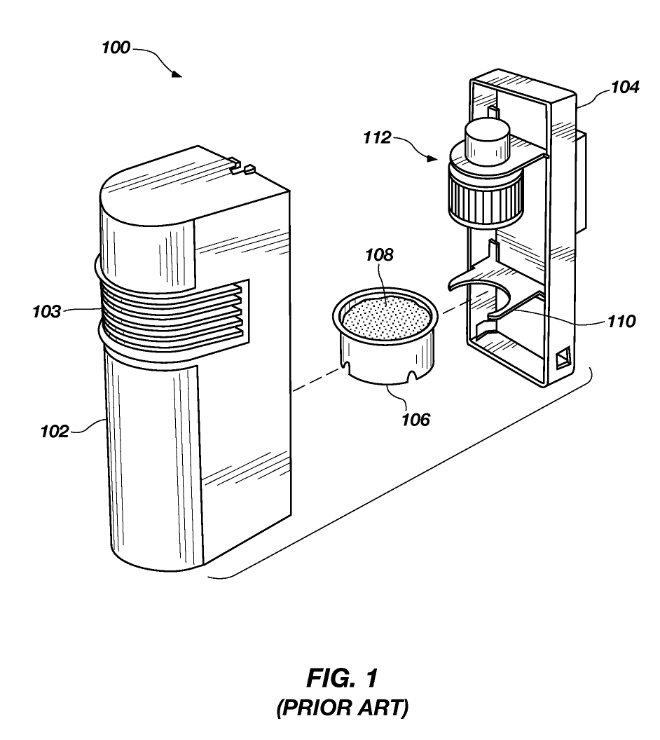

A prior art dispenser unit 100, shown in FIG. 1, comprises a front cover 102 having air vents 103, and a back piece 104. A disposable cartridge 106 having perfumed air freshener gel 108 is disposed in a cup holder 110. A battery-powered fan unit 112 is mounted over the cartridge 106 when it is positioned in holder 110. In use, the cover 102 is attached to the back piece 104 and the fan unit 112 is actuated. The resulting increased air flow over the open cartridge 106 causes an increase in evaporation of the air freshener gel 108, which is blown through the air vents 103 and into the surrounding air.

The present invention provides a mechanism for removing a foil seal that can be cut using a built-in safe cutting tool. The cutting tool is designed so that there are no exposed blades and it is safe for the user. The user may remove an expended refill cup and position it on top of the new refill cup. The user pushes the expended cup down over the refill cup and twists the expended cup causing substantial rotation of the expended cup relative to the refill cup. As a result, the foil of the new cup is cut open and caught by the cutting pieces in the bottom of the expended cup. When the expended cup is separated from the refill cup, the cut foil is pulled free of the refill cup, allowing access to the gel in the new cup. The new cup is then ready to be inserted into the fan dispenser and used as intended.

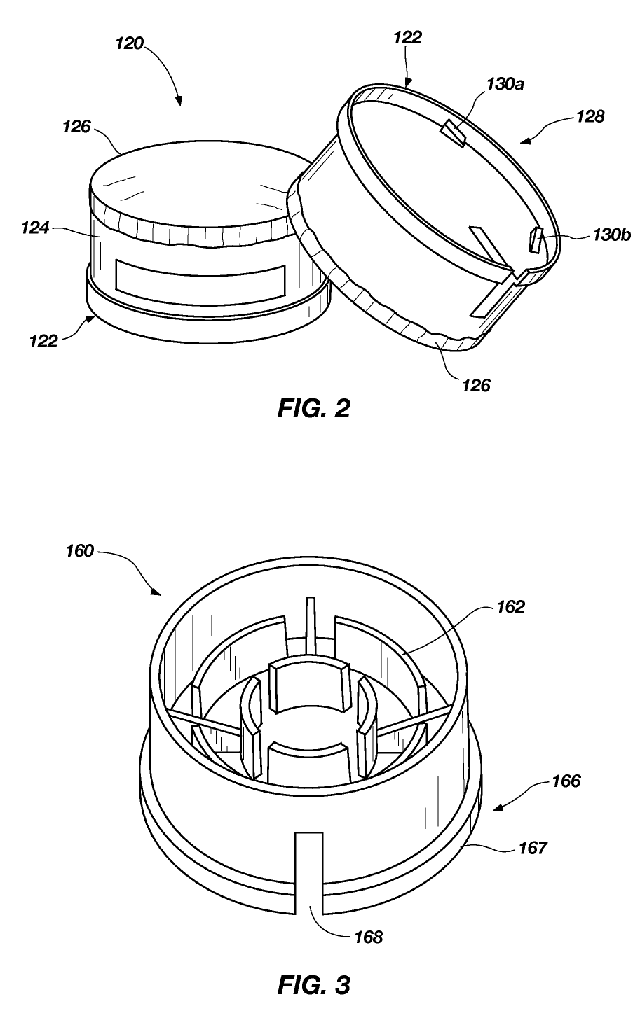

The present invention concerns a new structure of an air freshener refill cartridge for an air freshener dispenser. Referring to FIG. 2, one embodiment of the present invention shows two cartridges in the form of sealed refill cups 120. Each cup 122 has a circular shape 124 and is filled with an air-freshening gel (not shown here). The top of each cup 122 is tightly sealed by an aluminum foil 126. The bottom of each cup 128 has a unique structure comprising a plurality of cutting pieces, two of which are shown at 130a and 130b. The function of these cutting pieces will be subsequently shown and discussed.

Looking at FIG. 3, a refill cup 160 according to the present invention is shown, depleted of its gel (not shown here). A plurality of concentric and radial sections 162 are shown that facilitate gel evaporation by providing many cup surfaces each of which interfaces with the gel. As the gel evaporates and shrinks, spaces develop between each section 162 and the gel. Thus, in addition to the surface area provided at the flat surface of the gel, the spaces between each section 162 and the gel substantially increase the total evaporation surface area. This greatly enhances the air freshening capability of the new cartridge.

In FIG. 3, the base 166 of the depleted cup 160 has a skirt 167 with a greater diameter than the rest of cup 160, so that base 166 will fit over the top of another refill cup of the same dimensions. A slot 168 is shown for aligning the depleted cup 160 with a cartridge holder (not shown here) in an air freshening unit similar to the unit 100 shown in FIG. 1. The skirt 167 is slightly taller than the cutting blades, so that the blades are not exposed.

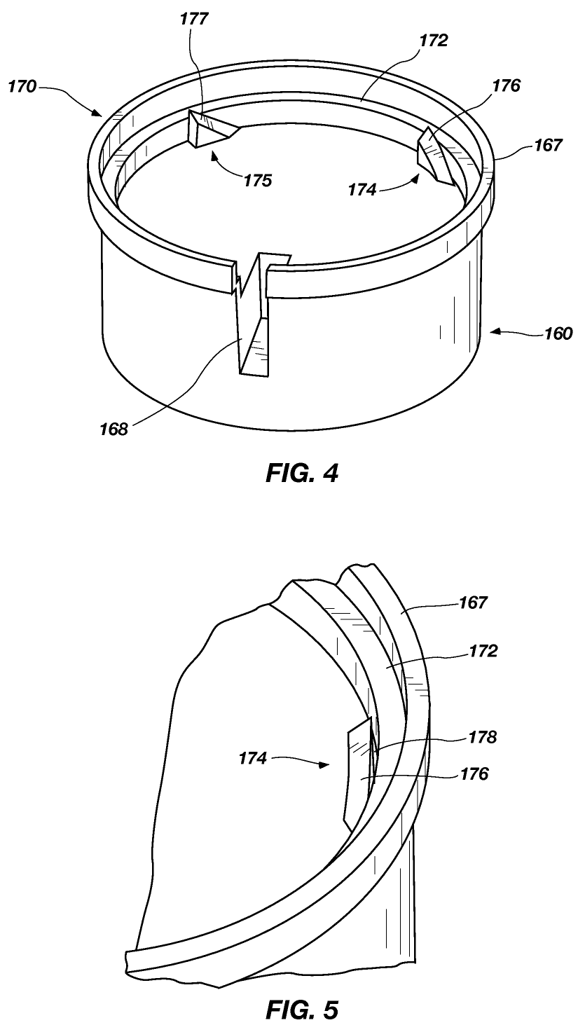

Referring to FIG. 4, the bottom 170 of cup 160 is shown. Below the skirt 167 is a circumferential ledge 172 for engaging the top of new refill cup of like structure (not shown here) so that the bottom 170 of the depleted cup 160 and top of the new refill cup mate with each other. Two blades 174 and 175 having inclined cutting surfaces 176 and 177 are circumferentially spaced from each other and disposed just inside of the ledge 172. Two other blades (not shown here) are similarly space around the bottom 170, just inside of the ledge 172.

FIG. 5 shows a close-up view of cutting piece 174 relative to ledge 172 and the skirt 167. A small space 178 is shown between cutting piece 174 and ledge 172. Space 178 is instrumental in trapping the foil of another refill cup (not shown here) so as to easily remove the foil from the cup.

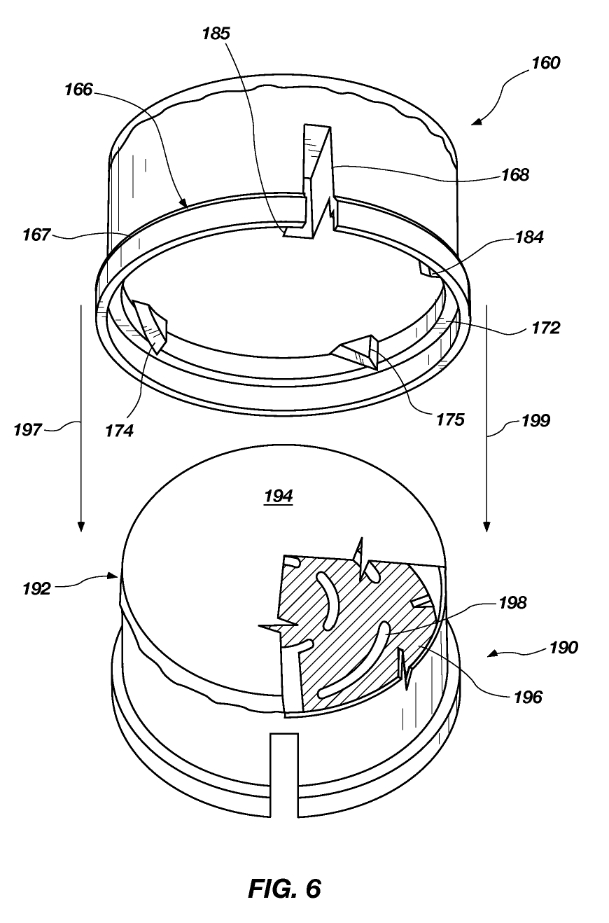

Referring now to FIG. 6, the operation of the disclosed embodiment of the present invention is shown. Depleted cup 160 is disposed over the top of a new refill cup 190 of like structure. A foil seal 194 covers the top 192 of the refill cup 190. The base 166 of depleted cup 160 is positioned to mate with the top 192 of new refill cup 190 so that blades 174 and 175, as well as partially viewed blades 184 and 185, will cut into the periphery of the foil seal 194. Beneath foil seal 194 is a gel 196 and the tops of a plurality of sections 198 of the type previously described herein. As shown by the arrows 197 and 199, the depleted cup 160 is meant to be moved downward toward new refill cup 190.

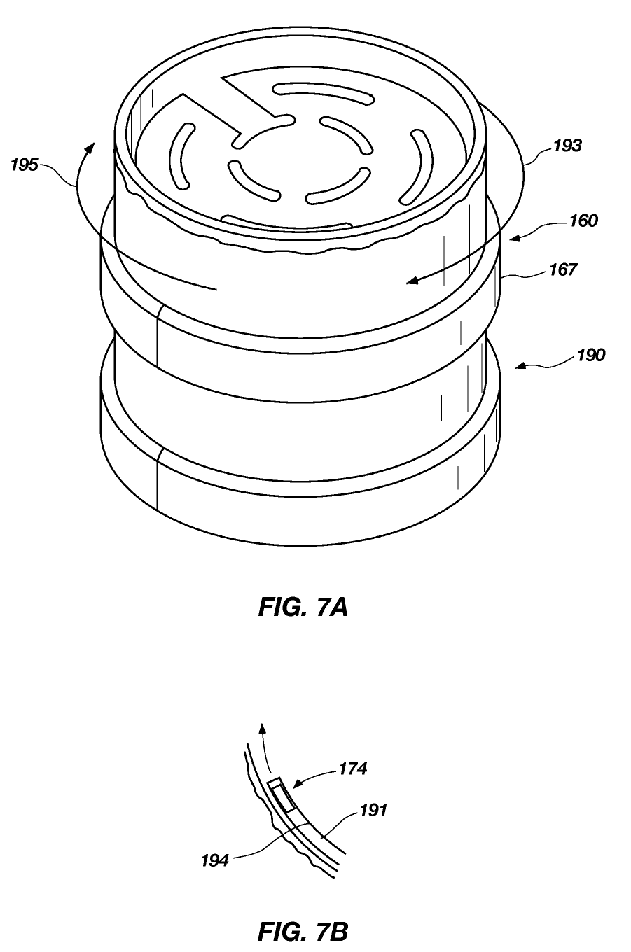

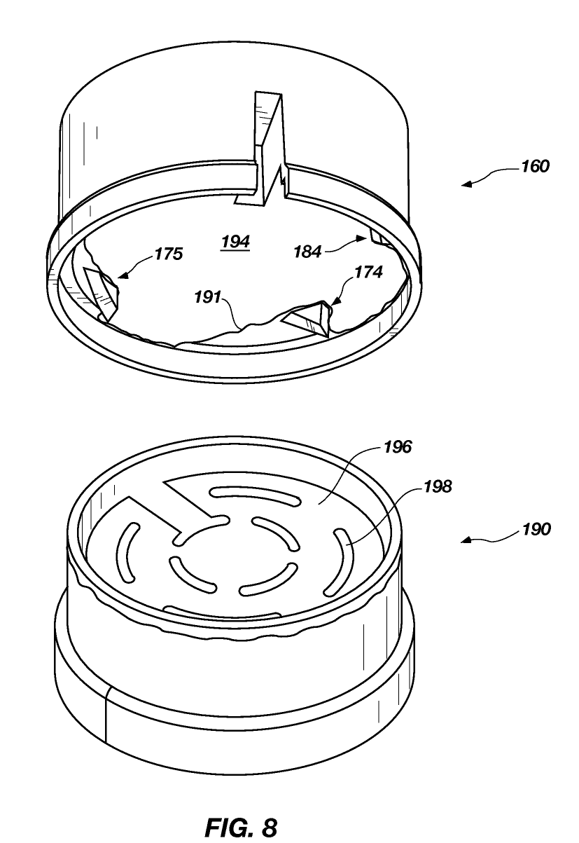

FIG. 7A shows the depleted cup 160 and new refill cup 190 in fully mated position with the skirt 167 of the depleted cup 160 entirely covering the top 192 of the new refill cup 190. The ledge 172 (not shown here) ensures that the depleted cup 160 may not be pushed too far down into the new refill cup 190. As shown by arrows 193 and 195, depleted cup 160 may be rotated clockwise or counter-clockwise relative to new refill cup 190 sufficient to cut into the foil seal 194 (not shown here). In one embodiment, the rotation of depleted cup 160 relative to new refill cup 190 is approximately 45 degrees to 90 degrees.

FIG. 7B is a close up view of cutting member 174. As depleted cup 160 is rotated clockwise or counter-clockwise with respect to new refill cup 190, cutting member 174 slices through foil seal 194 to leave an opening 191 around the periphery of foil seal 194.

Looking now at FIG. 8, depleted cup 160 has been removed from new refill cup 190. Foil seal 194 has been caught by teeth 174, 175, 184, 185 to detach from new refill cup 190 as depleted cup 160 is removed. The surface of gel 196 and the tops of a plurality of sections 198 are now exposed to enable the evaporation action of the gel to begin. Depleted cup 160 and removed foil seal 194 may be discarded or recycled.

As shown herein, the preferred embodiment of this invention enables the opening of refill cups without the use of knives or other sharp objects that might injure a user. The invention enables one to prepare freshener containers for use in a fast, easy, and safe manner.

It should be understood that the embodiment shown herein is only one embodiment of the invention, and many variations of the present embodiment will fall within the scope of the present invention. For example, rather than being made of plastic, the cups shown herein may be made partially or completely of metal or other suitable material. Other types, shapes and numbers of blades may be used beside those that are shown herein, as well as their positioning on the bottom of a cup. In addition, attachment members, such as magnetic means, rather than blades, may be used to attach to and remove the foil seal. Further, the foil seal may be replaced by other sealing means over the top of the gel, such as a metal lid. In addition, other types of air fresheners, including liquids and solids, may be used instead of gels. As used herein, the term “air freshener” refers to any type and composition of freshener, deodorant or other substance that may be used to clean the air, neutralize or mask odors, or to stimulate the olfactory senses.

It will be understood that the invention may be embodied in other specific forms by one of ordinary skill in the art without departing from the spirit, characteristics or coverage of the present invention. The present example and embodiment are to be considered to be illustrative and not restrictive, and the invention is not intended to be limited to the details of the described embodiments. Rather, the invention is defined by the claims, and as broadly as the prior art will permit.

The subject matter claimed is:

1. A container for containing an air freshener, comprising:
(a) a housing having a wall and a base for containing the air freshener, and having an opening for exposing the air freshener to the air;
(b) a removable cover disposed over the opening; and
(c) contacting means on the housing adapted to contact with an aperture of another container having a cover device over an aperture and adapted to remove the cover device from the aperture.

2. The container of claim 1, wherein said another container is of the same structure and dimensions as the container.

3. The container of claim 1, wherein the housing and base comprise the shape of a cup.

4. The container of claim 2, wherein the cup is closed at the bottom and sides and sealed at the top by the removable sealing device.

5. The container of claim 1, wherein the air freshener is a gel disposed in the housing.

6. The container of claim 1, wherein the removable cover is a sealing device to completely cover and seal the opening.

7. The container of claim 6, wherein the sealing device is an aluminum foil.

8. The container of claim 1, wherein the contacting means is an attachment device adapted for attaching to the cover device of the another container to remove the cover device from the aperture.

9. The container of claim 8, wherein the attachment device is a plurality of blades adapted to cut into and retain the cover device.

10. The container of claim 8, wherein the attachment device is adapted to attach to the cover device as the container is moved relative to said another container.

11. A container for containing an air freshener, comprising:
(a) a first cup adapted to contain the air freshener, having a closed bottom and a first open top exposing the air freshener to the air;
(b) a removable first seal covering the first open top to prevent loss of the air freshener prior to use; and
(c) an attachment member on the closed bottom for contact with a second cup to remove a second seal covering a second open top of the second cup.

12. The container of claim 11, wherein the structure of the second cup is the same as the structure of the first cup.

13. The container of claim 11, wherein the attachment member comprises a plurality of blades adapted to cut into and retain the second seal.

14. The container of claim 11, wherein the bottom of the first cup is adapted to mate to the second opening of the second cup.

15. The container of claim 11, wherein the first cup and the second cup are plastic.

16. The container of claim 11, wherein the second seal is aluminum foil.

17. The container of claim 11, wherein the attachment device is adapted to attach to the second seal as the first cup is rotated relative to the second cup.

18. A method of using a first container to remove a cover on a second container, comprising:
(a) contacting a bottom of the first container with a top of the second container;
(b) moving the first container relative to the second container, so that attachment means on the first container attaches to the cover of the second container;
(c) removing the first container from the second container, thereby removing the cover from the second container.

19. The method of claim 18 wherein the moving step comprises rotating the first container relative to the second container.

20. The method of claim 19, wherein the step of removing the cover from the second container comprises removing a seal from the second container.

21. The method of claim 20, wherein the attachment means is a plurality of blades, and wherein the rotating step comprises cutting and retaining the seal of the second container using the plurality of blades.

22. The method of claim 18, wherein the contacting step comprises mating first structure on the bottom of the first container with second structure on the top of the second container.

23. A container for containing an air freshener, comprising:
(a) a cup adapted to contain the air freshener, having a closed bottom and an open top exposing the air freshener to the air;
(b) a seal over the open top to seal the air freshener from evaporation or vaporization until ready for use; and
(c) a plurality of members disposed in the cup to provide for additional surface area for the air freshener to evaporate or vaporize after the seal has been removed.

24. The container of claim 22, wherein the air freshener is a gel or solid.

25. The container of claim 24, wherein the plurality of members are disposed in the gel or solid, so that as the gel or solid evaporates or vaporizes, a substantial amount of additional surface area of the gel or solid is exposed for further evaporation or vaporization.
